(19) United States
(12) Patent Application Publication
(10) Pub. No.: US 2003/0154988 A1
(43) Pub. Date: Aug. 21, 2003

(54) Intra-bronchial device that provides a medicant intra-bronchially to the patient

(75) Inventors: Lauri J. DeVore, Seattle, WA (US); Richard O. Shea, Kenmore, WA (US); John H. Wang, Sammamish, WA (US); Hugo X. Gonzalez, Woodinville, WA (US); Steven Chase Springmeyer, Bellevue, WA (US)

Correspondence Address: GRAYBEAL, JACKSON, HALEY LLP, 155 - 108TH AVENUE NE, SUITE 350, BELLEVUE, WA, 98004-5901, US

(73) Assignee: Spiration, Inc.

(21) Appl. No.: 10/178,073

(22) Filed: Jun. 21, 2002

Related U.S. Application Data
(63) Continuation-in-part of application No. 10/081,712, filed on Feb. 21, 2002.

(51) Int. Cl. A61M 16/00

(52) U.S. Cl. 128/207.15; 604/96.01

(57) ABSTRACT

An intra-bronchial device provides a medicant intra-bronchially. The medicant may be used for controlling biological interaction of an intra-bronchial obstruction device with the patient, to treat a disease or condition of the lungs such as pneumonia or lung cancer, or to treat a systemic disease or condition. The medicant is provided by associating a medicant with the intra-bronchial device, either before, at the time of placement, or after placement. The medicant may overlie at least a portion of the intra-bronchial device, be absorbed into at least a portion of the intra-bronchial device, or be carried in a chamber. The intra-bronchial device may further include an absorptive member, and the medicant is absorbed by the absorptive member.

### RELATED APPLICATION

This application is a continuation-in-part of and claims priority based on an application entitled “INTRA-BRONCHIAL OBSTRUCTING DEVICE THAT CONTROLS BIOLOGICAL INTERACTION WITH THE PATIENT” filed Feb. 21, 2002, application Ser. No. 10/081,712.

### BACKGROUND OF THE INVENTION

The present invention is generally directed to a device, system, and method that provides a medicant intra-bronchially to a patient by an intra-bronchial device placed in an air passageway. The present invention is more particularly directed to an intra-bronchial device that provides a medicant that controls biological interaction of the device with the patient, or that provides a medicant intra-bronchially that treats diseases and conditions of the patient, particularly those associated with the lungs such as pneumonia and lung cancer.

An aspect of the invention is directed toward treating Chronic Obstructive Pulmonary Disease (COPD), which has become a major cause of morbidity and mortality in the United States over the last three decades. COPD is characterized by the presence of airflow obstruction due to chronic bronchitis or emphysema. The airflow obstruction in COPD is due largely to structural abnormalities in the smaller airways. Important causes are inflammation, fibrosis, goblet cell metaplasia, and smooth muscle hypertrophy in terminal bronchioles.

The incidence, prevalence, and health-related costs of COPD are on the rise. Mortality due to COPD is also on the rise. In 1991, COPD was the fourth leading cause of death in the United States and had increased 33% since 1979.

COPD affects the patient's whole life, producing increasing disabilities. It has three main symptoms: cough; breathlessness; and wheeze. At first, breathlessness may be noticed when running for a bus, digging in the garden, or walking uphill. Later, it may be noticed when simply walking in the kitchen. Over time, it may occur with less and less effort until it is present all of the time.

COPD is a progressive disease and currently has no cure. Current treatments for COPD include the prevention of further respiratory damage, pharmacotherapy, and surgery. Each is discussed below.

The prevention of further respiratory damage entails the adoption of a healthy lifestyle. Smoking cessation is believed to be the single most important therapeutic intervention. However, regular exercise and weight control are also important. Patients whose symptoms restrict their daily activities or who otherwise have an impaired quality of life may require a pulmonary rehabilitation program including ventilatory muscle training and breathing retraining. Long-term oxygen therapy may also become necessary.

Pharmacotherapy may include bronchodilator therapy to open up the airways as much as possible or inhaled beta-agonists. For those patients who respond poorly to the foregoing or who have persistent symptoms, ipratropium bromide may be indicated. Further, courses of steroids, such as corticosteroids, may be required. Lastly, antibiotics may be required to prevent infections and influenza and pneumococcal vaccines may be routinely administered. Unfortunately, there is no evidence that early, regular use of pharmacotherapy will alter the progression of COPD.

About 40 years ago, it was first postulated that the tethering force that tends to keep the intrathoracic airways open was lost in emphysema and that by surgically removing the most affected parts of the lungs, the force could be partially restored. Although the surgery was deemed promising, the procedure was abandoned. The lung volume reduction surgery (LVRS) was later revived. In the early 1990's, hundreds of patients underwent the procedure. However, the number of procedures declined because Medicare stopped reimbursing for LVRS. The procedure is currently under review in controlled clinical trials. Preliminary data indicates that patients benefited from the procedure in terms of an increase in forced expiratory volume, a decrease in total lung capacity, and a significant improvement in lung function, dyspnea, and quality of life. Improvements in pulmonary function after LVRS have been attributed to at least four possible mechanisms; enhanced elastic lung recoil, correction of ventilation/perfusion mismatch, improved efficiency of respiratory musculature, and improved right ventricular filling.

Lastly, lung transplantation is also a therapeutic option. Today, COPD is the most common diagnosis for which lung transplantation is considered. Unfortunately, this consideration is given for only those with advanced COPD. Given the limited availability of donor organs, lung transplant is far from being available to all patients.

The inventions disclosed and claimed in U.S. Pat. Nos. 6,258,100 and 6,293,951, both of which are incorporated herein by reference, provide an improved therapy for treating COPD. The therapy includes non-surgical apparatus and procedures for reducing lung volume by permanently obstructing the air passageway that communicates with the portion of the lung to be collapsed. An obstruction device is placed in the air passageway that prevents inhaled air from flowing into the portion of the lung to be collapsed. This provides lung volume reduction with concomitant improved pulmonary function without the need for surgery. Various other apparatus and techniques may exist for permanently obstructing the air passageway.

Obstructing devices in an air passageway may contribute to a biological interaction with the patient, such as infection, inflammation, tissue granulation, and biological reaction. Furthermore, biological interaction may adversely affect the functionality of the obstructing device by creating unwanted buildup of biological material on the device, and compromising the ability of the obstructing device to remain in position.

Another aspect of the invention is directed toward targeted intra-bronchial delivery of a medicant that treats diseases and conditions of the patient, particularly those associated with the lungs such as pneumonia and lung cancer. Treatment of certain lung diseases and conditions will benefit from targeted intra-bronchial delivery of a medicant into the involved regions. Treatment will be further benefited if the medicant is generally confined to the involved regions. For example, treatment of a disease such as pneumonia will benefit by being able to deliver an antibiotic to the specific lung region involved. Furthermore, treatment of lung cancer will benefit by non-invasive brachytherapy. However, no device, system, or method presently exists that provides for non-invasive targeted intra-bronchial delivery of a medicant to specific lung regions.

In view of the foregoing, there is a need in the art for a new and improved device and method for obstructing an air passageway that controls the biological interaction between the device and the patient. There is further a need for a new and improved device, and method for targeted intra-bronchial delivery of a medicant to specific lung regions. The present invention is directed to providing such an improved apparatus and method for intra-bronchial delivery of a medicant to specific sites in the lungs, such as the location of an intra-bronchial device treating COPD or a diseased lung region.

### SUMMARY OF THE INVENTION

The present invention provides an intra-bronchial device that controls biological interaction of the device with the patient. The intra-bronchial device is adapted to be placed in an air passageway of a patient to collapse a lung portion communicating with the air passageway. The device includes an obstructing member that prevents air from being inhaled into the lung portion to collapse the lung portion, and a medicant carried by the obstructing member. The medicant may overlie at least a portion of the obstructing member, or the medicant may be absorbed in at least a portion of the obstructing member. The obstructing member may further include an absorptive member, and the medicant is absorbed by the absorptive member.

The medicant may be selected from a group consisting of tissue growth inhibitors, tissue growth enhancers, anti-microbial agents such as antibiotic agents or antibacterial agents, anti-inflammatory agents, and biological reaction inhibitors. The medicant may be arranged to control biological interaction over a period of time.

In accordance with a further embodiment, the present invention provides an intra-bronchial device and a medicant that controls biological interaction of the device with the patient. The intra-bronchial device is adapted to be placed in an air passageway of a patient to collapse a lung portion communicating with the air passageway. It includes an obstructing member that prevents air from being inhaled into the lung portion to collapse the lung portion, and a cavity in the obstructing member carrying the medicant. The cavity may further include an absorptive member, and the medicant is absorbed by the absorptive member.

The invention further provides a method of reducing the size of a lung of a patient using an intra-bronchial device while controlling biological interaction of the device with the patient. The method includes the step of providing an intra-bronchial device that precludes air from being inhaled through an air passageway into a lung portion to be reduced in size when inserted into the air passageway communicating with the portion of the lung. The method also includes the step of associating a medicant that controls the biological interaction with the intra-bronchial device. The method further includes the step of inserting the intra-bronchial device in the air passageway. The step of associating the medicant with the intra-bronchial device may be performed before the step of implanting the device. The step of associating the medicant with the intra-bronchial device may include overlying at least a portion of the intra-bronchial device with the medicant. In an alternative embodiment, the step of associating the medicant with the intra-bronchial device includes impregnating at least a portion of the intra-bronchial device with the medicant. The method may also include the further steps of providing a cavity in the intra-bronchial device for receiving the medicant, and providing the cavity with the medicant.

In yet another embodiment, the method further includes the steps of providing a cavity in the intra-bronchial device for receiving the medicant, and associating the medicant with the cavity. The cavity may include an absorptive member, and the step of associating medicant with the intra-bronchial device includes absorption of the medicant by the absorptive member. The step of associating the medicant with the intra-bronchial device may be performed before the step of implanting the device.

In accordance with another embodiment, the invention provides an intra-bronchial device that provides a medicant intra-bronchially to a patient. The device includes an intra-bronchial member adapted to be placed in an air passageway, and a medicant carried on the intra-bronchial member. The intra-bronchial device may include a cavity in the intra-bronchial member, and the medicant is carried in the cavity. The medicant may be arranged for delivery to a lung portion communicating with the air passageway. The medicant may be selected from a group consisting of antibacterial agents, antiviral agents, anthelmintic agents, anti-inflammatory agents, antitumor agents, radioprotective agents, antioxidant agents, adrenergic agents, hormonal agents, and radioactive branchytherapy material. The intra-bronchial member may be arranged to preclude air movement in at least one direction. The medicant may overlie at least a portion of the intra-bronchial member, may be imbedded in at least a portion of the intra-bronchial member, or may be absorbed in at least a portion of the intra-bronchial member.

In accordance with still another embodiment of the invention, the invention provides an intra-bronchial device adapted to be placed in an air passageway and that provide a medicant to a patient. The intra-bronchial device includes an obstructing member that prevents air from being exhaled from the lung portion communicating with the air passageway, and a medicant carried on the obstructing member. The medicant may be arranged for delivery to the lung portion, and may be carried on a portion of the obstructing member exposed to the lung portion. The obstructing member when deployed in the air passageway may substantially preclude released medicant from moving proximal to the obstructing member. The medicant may overlie, be imbedded in, co-mixed with, or absorbed in at least a portion of the obstructing member. The obstructing member may include an absorptive member and the medicant may be absorbed by the absorptive member.

Another embodiment of the invention provides an intra-bronchial device adapted to be placed in an air passageway and provide a medicant to a patient. The intra-bronchial device includes an obstructing member that prevents air from being exhaled from the lung portion communicating with the air passageway, a medicant, and a cavity in the obstructing member carrying the medicant. The cavity may further include an absorptive member and the medicant may be absorbed by the absorptive member, and may include a cover having an orifice affecting release of the medicant. The medicant may be exposed to the lung portion. The obstructing member when deployed in the air passageway may substantially preclude released medicant from moving proximal to the obstructing member.

Yet another embodiment of the invention provides an intra-bronchial device adapted to be placed in an air passageway and provide a medicant to a patient. The intra-bronchial device includes an obstructing member that prevents air from being exhaled from the lung portion communicating with the air passageway, a medicant, and a support structure that is associated with the obstructing member and that carries the medicant.

An additional further embodiment of the invention provides a method of providing a medicant to a patient using an intra-bronchial device. The method includes the steps of providing an intra-bronchial device for insertion into an air passageway in communication with a lung portion, associating a medicant with the intra-bronchial device, and inserting the intra-bronchial device in the air passageway. The intra-bronchial device may preclude air from being exhaled through the air passageway when inserted into the air passageway. The medicant may be an agent for treating a disease of the lungs, and the medicant may be provided to treat a disease in the lung portion. The medicant may be an agent for treating pneumonia, and the medicant may be provided to treat pneumonia in the lung portion. The medicant may be a radioactive material for treating cancer, and the medicant is provided to treat a cancer, which may be in the lung portion.

In yet a further embodiment, the invention provides a device for reducing the size of a lung of a patient. The device includes obstructing means for obstructing an air passageway communicating with a portion of the lung to be reduced in size, the obstructing means being dimensioned for insertion into the air passageway and for precluding air from being inhaled through the air passageway into the lung portion, and a means for controlling biological interaction of the obstructing means with the patient.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify identical elements, and wherein:

FIG. 1 is a simplified sectional view of a thorax illustrating a healthy respiratory system;

FIG. 2 is sectional view similar to FIG. 1 but illustrating a respiratory system suffering from COPD, and an initial step in placing an obstructing member;

FIG. 3 illustrates a further step in a method for placement of an obstructing member in a bronchial sub-branch;

FIG. 4 is a perspective view, partly in section, illustrating an obstructing member positioned in an air passageway for sealing the lung portion;

FIG. 5 is a longitudinal view of an air passageway illustrating additional details of an obstructing member inserted into an air passageway and preventing air from being inhaled;

FIG. 6 is a longitudinal section view illustrating an obstructing member inserted in an air passageway and carrying a medicant;

FIG. 7 is a longitudinal section view illustrating an obstructing member having a cavity for carrying medicant according to an alternative embodiment of the invention;

FIG. 8 illustrates an obstructing member similar to FIG. 7 with an orifice included to affect release of medicant;

FIG. 9 is a longitudinal section view illustrating an obstructing member having a cavity that includes an absorptive member for carrying a medicant according to another alternative embodiment of the invention;

FIGS. 10 and 11 illustrate provision of localized control of biological interaction according to a further alternative embodiment of the invention;

FIGS. 12 and 13 illustrate the use of a medicant to encourage a targeted expression of a biological response for an anchored intra-bronchial device in accordance with the present invention;

FIG. 14 illustrates the use of a medicant to encourage a targeted expression of a biological response for another embodiment of an anchored intra-bronchial device, in accordance with the present invention; and

FIG. 15 illustrates a longitudinal cross-section view of the intra-bronchial device of FIGS. 10 and 11 placed in an air passageway to provide a medicant to a patient, in accordance with the present invention.

### DETAILED DESCRIPTION OF THE PREFERRED EMBODIMENTS

In the following detailed description of exemplary embodiments of the invention, reference is made to the accompanying drawings that form a part hereof. The detailed description and the drawings illustrate specific exemplary embodiments by which the invention may be practiced. These embodiments are described in sufficient detail to enable those skilled in the art to practice the invention. It is understood that other embodiments may be utilized, and other changes may be made, without departing from the spirit or scope of the present invention. The following detailed description is therefore not to be taken in a limiting sense, and the scope of the present invention is defined by the appended claims.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein. Additionally, throughout the specification, claims, and drawings, the term “proximal” means nearest the trachea, and “distal” means nearest the bronchioli.

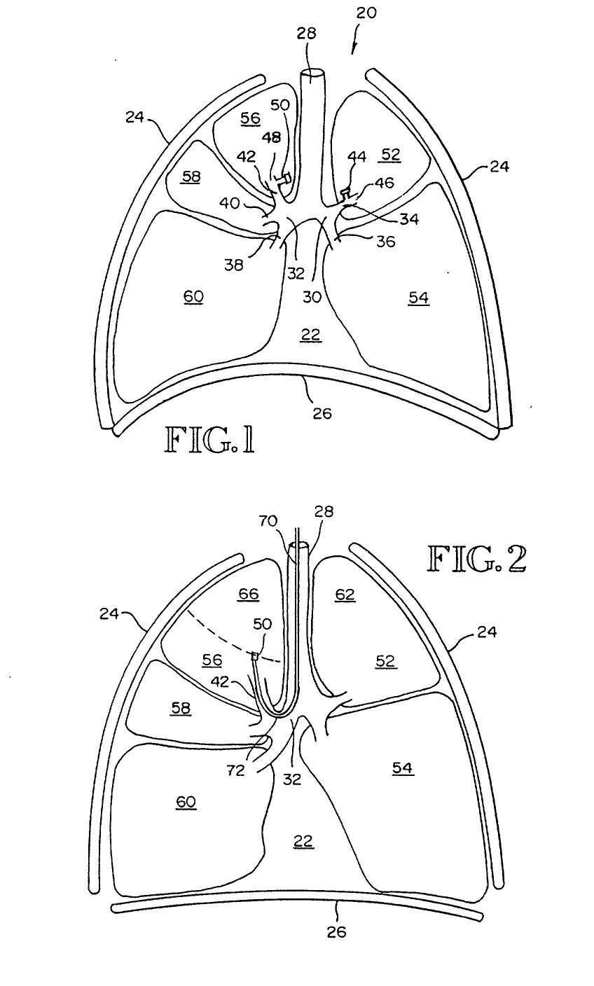

FIG. 1 is a sectional view of a healthy respiratory system. The respiratory system 20 resides within the thorax 22 which occupies a space defined by the chest wall 24 and the diaphragm 26.

The respiratory system 20 includes the trachea 28, the left mainstem bronchus 30, the right mainstem bronchus 32, the bronchial branches 34, 36, 38, 40, and 42 and sub-branches 44, 46, 48, and 50. The respiratory system 20 further includes left lung lobes 52 and 54 and right lung lobes 56, 58, and 60. Each bronchial branch and sub-branch communicates with a respective different portion of a lung lobe, either the entire lung lobe or a portion thereof. As used herein, the term “air passageway” is meant to denote either bronchi or bronchioles, and typically means a bronchial branch or sub-branch which communicates with a corresponding individual lung lobe or lung lobe tissue portion to provide inhaled air thereto or conduct exhaled air therefrom.

Characteristic of a healthy respiratory system is the arched or inwardly arcuate diaphragm 26. As the individual inhales, the diaphragm 26 straightens to increase the volume of the thorax 22. This causes a negative pressure within the thorax. The negative pressure within the thorax in turn causes the lung lobes to fill with air. When the individual exhales, the diaphragm returns to its original arched condition to decrease the volume of the thorax. The decreased volume of the thorax causes a positive pressure within the thorax that in turn causes exhalation of the lung lobes.

FIG. 2 illustrates a respiratory system suffering from COPD. Here it may be seen that the lung lobes 52, 54, 56, 58, and 60 are enlarged and that the diaphragm 26 is not arched but substantially straight. Hence, this individual is incapable of breathing normally by moving the diaphragm 28. Instead, in order to create the negative pressure in the thorax 22 required for breathing, this individual must move the chest wall outwardly to increase the volume of the thorax. This results in inefficient breathing causing these individuals to breathe rapidly with shallow breaths.

It has been found that the apex portions 62 and 66 of the upper lung lobes 52 and 56, respectively, are most affected by COPD. Hence, bronchial sub-branch obstructing devices are generally employed for treating the apex 66 of the right, upper lung lobe 56. However, as will be appreciated by those skilled in the art, the present invention may be applied to any lung portion without departing from the present invention. As will be further appreciated by those skilled the in art, the present invention may be used with any type of obstructing member to permit mucociliary transport. The inventions disclosed and claimed in U.S. Pat. Nos. 6,258,100 and 6,293,951, both of which are incorporated herein by reference, provide an improved therapy for treating COPD by obstructing an air passageway using an intra-bronchial device, such as a valve or plug. The present invention may be used with the apparatus, system, and methods of these patents as will be briefly described in conjunction with the disclosure of the preferred embodiments of the present invention.

The insertion of an obstructing member treats COPD by deriving the benefits of lung volume reduction surgery without the need of performing the surgery. The treatment contemplates permanent partial or complete collapse of a lung portion to reduce lung mass. This leaves extra volume within the thorax for the diaphragm to assume its arched state for acting upon the remaining healthier lung tissue. As previously mentioned, this should result in improved pulmonary function due to enhanced elastic recoil, correction of ventilation/perfusion mismatch, improved efficiency of respiratory musculature, and improved right ventricle filling.

FIG. 2 also illustrates a step in COPD treatment using an intra-bronchial device having an obstructing member using a catheter or bronchoscope. The invention disclosed herein is not limited to use with the particular method illustrated herein. Catheter 70 may be used alone to perform the insertion, may be extended from a bronchoscope, or used in conjunction with a bronchoscope. For purposes of this description, the insertion will be described with reference to only the catheter 70. Treatment is initiated by feeding a conduit, such as a catheter 70 down the trachea 28, into the right mainstem bronchus 32, into the bronchial branch 42 and into and terminating within the sub-branch 50. The sub-branch 50 is the air passageway that communicates with the lung portion 66 to be treated. The catheter 70 is preferably formed of flexible material such as polyethylene. Also, the catheter 70 is preferably preformed with a bend 72 to assist the feeding of the catheter from the right mainstem bronchus 32 into the bronchial branch 42, or could be deformed to conform to different curvature and angles of a bronchial tree.

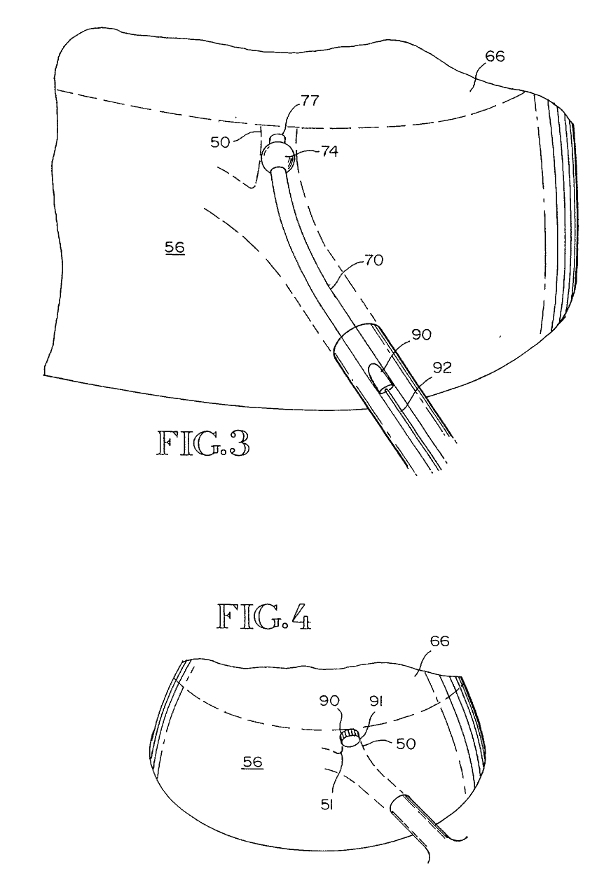

FIG. 3 illustrates a further step in a method for inserting an obstructing member 90 of an intra-bronchial device in a bronchial sub-branch using a catheter or a bronchoscope. Catheter 70 may include an optional inflatable sealing member 74 for use with a vacuum to collapse lung portion 66 prior to insertion of obstructing member 90. The obstructing member 90 may be formed of resilient or collapsible material to enable the obstructing member 90 to be fed through the conduit 70 in a collapsed state. The stylet 92 is used to push the obstructing member 90 to the end 77 of the catheter 70 for inserting the obstructing member 90 within the air passageway 50 adjacent to the lung portion 66 to be permanently collapsed. Optional sealing member 74 is withdrawn after obstructing member 90 is inserted.

FIG. 4 illustrates the obstructing member 90 inserted in air passageway 50. Obstructing member 90 has expanded upon placement in the air passageway 50 to prevent air from being inhaled into the lung portion. This causes the lung portion 66 to be maintained in a permanently collapsed state. The obstructing member 90 may be any shape and composed of any material suitable for accomplishing its purpose. For example, possible shapes include spherical, cylindrical, and conical. By way of further example, obstructing member 90 may be a solid member, a composition of materials, or a membrane.

More specifically, the obstructing member 90 has an outer dimension 91, and when expanded, enables contact with the air passageway inner dimension 51. This seals the air passageway upon placement of the obstructing member 90 in the air passageway 50 for maintaining the lung portion 66 in the collapsed state. According to an embodiment of the invention, the intra-bronchial device, such as obstructing member 90, may include an anchor that anchors the intra-bronchial device within the air passageway as disclosed in “REMOVABLE ANCHORED LUNG VOLUME REDUCTION DEVICES AND METHODS” filed Mar. 20, 2002, application Ser. No. 10/104,487; “REMOVABLE ANCHORED LUNG VOLUME REDUCTION DEVICES AND METHODS” filed Apr. 16, 2002, application Ser. No. 10/124,790; and “REMOVABLE ANCHORED LUNG VOLUME REDUCTION DEVICES AND METHODS” filed May 17, 2002, application Ser. No. 10/150,547, all of which are incorporated herein by reference and collectively referred to as “Applications for Anchored Devices.”

Treating COPD and other diseases and conditions of the lungs according to an embodiment of the invention may involve obstructing a plurality of air passageways with obstructing members. In addition, redundant air passageway obstructions may be used. For example, a fifth-generation bronchial segment and its multiple sixth-generation bronchial subdivisions may each be obstructed to collapse a lung portion communicating with the fifth-generation bronchial segment.

Alternatively, the lung portion 66 may be collapsed using vacuum prior to placement of obstructing member 90, or it may be collapsed by sealing the air passageway 50 with obstructing member 90. Over time, the air within the lung portion -66 will be absorbed by the body and result in the collapse of lung portion 66. Alternatively, obstructing member 90 may include a one-way valve allowing air to escape from lung portion 66. Lung portion 66 will then collapse, and the valve will prevent air from being inhaled.

A function of the intra-bronchial device disclosed and claimed in the specification, including the detailed description and the claims, is described in terms of collapsing a lung portion communicating with an air passageway. In some lungs, a portion of a lung may receive air from collateral air passageways. Obstructing one of the collateral air passageways may reduce the volume of the lung portion communicating with the air passageway, but not completely collapse the lung portion as that term may be generally understood. As used herein, the meaning of “collapse” includes a complete collapse, a partial collapse, and a reduction in volume of a lung portion.

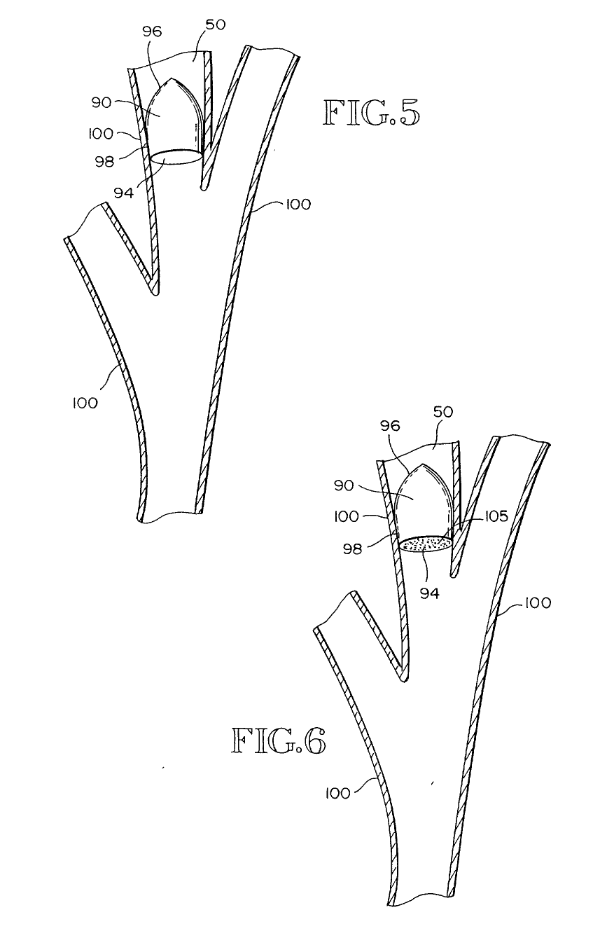

FIG. 5 is a longitudinal view of an air passageway illustrating additional details of an obstructing member inserted into an air passageway and preventing air from being inhaled. In this embodiment, obstructing member 90 generally has conical configuration, and may be hollow. More specifically, the obstructing member 90 includes a periphery that renders it generally circular at its base, referred to herein as generally circular base 94. The obstructing member 90 further includes a circumferential, generally conical sidewall 96 that extends from the outer periphery of generally circular base 94. The sidewall 96 has an exterior perimeter surface 98 that defines the outer periphery of the obstructing member 90. The obstructing member 90 is arranged so that a portion of its exterior perimeter surface 98 contacts bronchial wall 100 to form a seal that precludes air from moving past obstructing member 90.

Inserting obstructing member 90 into air passageway 50 may result in biological interaction with the patient that adversely effects the patient or the performance of obstructing member 90. Possible interactions include tissue granulation, infection, inflammation, and fibrotic response. For example, the presence of obstructing member 90 in the air passageway 50 may invoke the body's healing process. The healing process may involve tissue granulation and connective tissue projections that could interfere with the intra-bronchial device. The tissue granulation may begin on insertion of obstructing member 90, or sometime later. By way of another example, the presence of obstructing member 90 may result in a potential for infection or inflammation, which could occur on insertion of obstructing member 90 or sometime later. In a further example, the presence of obstructing member 90 in the air passageway 50 may invoke the patient's fibrotic response, which could interfere with obstructing member 90.

FIG. 6 is a longitudinal section view illustrating an obstructing member of an intra-bronchial device inserted in an air passageway and carrying a medicant, according to an embodiment of the invention. The medicant is selected according to the treatment objective and biological action desired, which may include controlling biological interaction or intra-bronchial delivery of a medicant to the patient that provides a biological action. For purposes of clarity in the specifications and drawings, embodiments of the invention are generally illustrated with obstructing member 90 as the only element of the intra-bronchial device. Alternative embodiments of an intra-bronchial device according to an aspect of the invention may include additional elements, such as structural members, anchors, and other elements, which are disclosed in the Applications for Anchored Devices.

In accordance with a broad aspect of the present invention, a medicant is associated with an obstructing member of an intra-bronchial device for release or presentment to the patient. The term “medicant” is broadly used in the specification herein, including the description and claims. “Medicant” includes anything presented for treatment, curing, mitigating, or preventing deleterious conditions in humans and animals. “Medicant” also includes anything used in medical diagnosis, or restoring, correcting, or modifying physiological functions. The medicant may be presented to control biological interaction of the intra-bronchial device with the patient, or to treat a disease or condition in the patient, particularly those associated with the lungs, such as pneumonia or lung cancer. The medicant may be associated with the obstructing member in many different ways. It may be carried on proximal, distal, or both proximal and distal portions of the device as may be required by the intended biological action and limitations of the selected medicant. FIG. 6, for example, illustrates an embodiment where medicant 105 overlies the surface of generally circular base 94 of obstructing member 90. If obstructing member 90 is a membrane or generally hollow structure, medicant 105 may be carried by overlayment on any suitable surface or surfaces, including an interior surface. Medicant 105 may be associated with all or any portion of the obstructing member 90 in any manner known to those skilled in the art, and as required by the biological action desired and the limitations of the selected medicant 105. Association methods include overlayment, absorption, and imbedding, which may be by any method known to those in the art, including spraying, dipping, ion implantation, and painting.

Alternative embodiments of the invention may include associating medicant 105 by impregnation, co-mixing, or absorption into obstructing member 90 in any manner known to those skilled in the art, and as required by biological action desired and the limitations of the selected medicant 105. For example, an anti-microbial medicant 105 may be absorbed into at least a portion of obstructing member 90.

Still further, the medicant may be carried on an element of an intra-bronchial device, which in turn is carried by obstructing member 90. Such elements may include structural members, or anchors for example.

The medicant 105 carried by, or associated with, the obstructing member 90 may be selected from any class suitable for the biological action desired. For example, if the desired biological action is controlling biological interaction of the intra-bronchial device with the patient, several classes of medicants may be used. These classes include tissue growth inhibitors, such as paclitaxel sold under the trademark Taxol™ of the Bristol-Meyers Co., that may stop cells from dividing and growing on obstructing member 90 so that they eventually die; tissue growth enhancers such as tissue growth factors; anti-microbial agents to prevent or resist seeding of bacteria on obstructing member 90, such as an anti-microbial compound that permits a continuous, controlled release of ionic silver over an extended time period sold as AgION™ of Agion Technologies, L.L.C.; biological reaction inhibitors, such as parylene, a common generic name for a unique series of polymers based on paraxylene that enhance biotolerence of medical devices used within the body, such as obstructing member 90; and antibiotics to control any infections associated with the obstructing member 90.

By way of further example, if the desired biological action is providing a medicant that treats a disease or condition of the patient, particularly those associated with lungs, several additional classes of medicants may be associated. These additional classes include antibiotics, such as antibiotics used to treat acute or chronic pneumonia, such as penicillin, ceftriaxone, tobramycin, vancomycin; antibacterial agents, antiviral agents, anthelmintic agents, anti-inflammatory agents, antitumor agents, radioprotective agents, antioxidant agents, adrenergic agents, and hormonal agents. If the desired biological action is brachytherapy treatment of cancer in lung or nearby tissue, the medicant may include radioactive material in the form of radioactive seeds providing radiation treatment directly into the tumor or close to it.

Further, the medicant 105 may be selected or arranged to control biological activity over time. The medicant may be associated with obstructing member 90 either before it is inserted into air passageway 50 or after, or renewed after insertion. Medicant provision may be terminated by removing the intra-bronchial device from the patient as disclosed in the Applications for Anchored Devices.

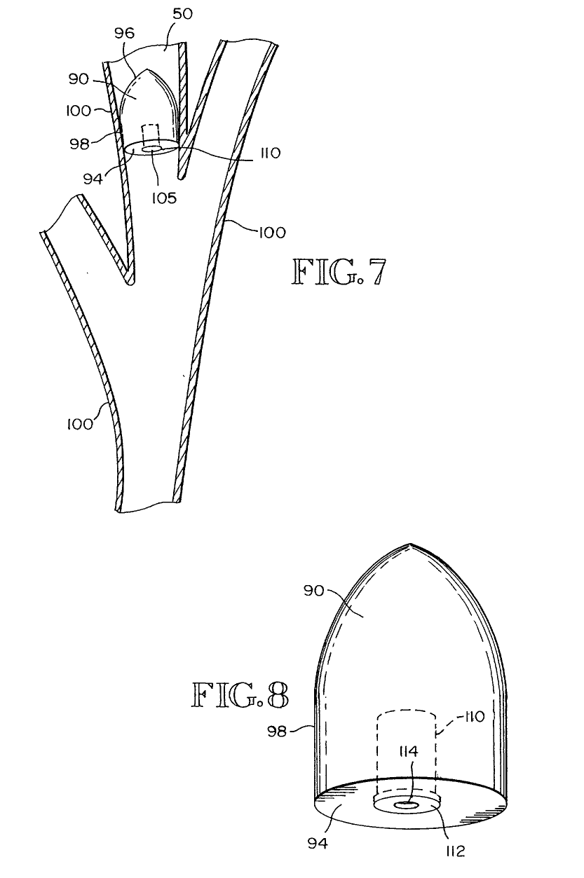

FIG. 7 is a longitudinal section view illustrating an obstructing member of an intra-bronchial device having a cavity for carrying medicant, according to an alternative embodiment of the invention. Obstructing member 90 includes a cavity 110 that carries medicant 105. While cavity 110 is illustrated in FIG. 7 as being cylindrical in configuration, it may be of any shape. Radioactive seeds may be carried in cavity 110. As described above, a plurality of intra-bronchial devices may be placed in a lung portion thus allowing providers to group radioactive seeds in a manner similar to that used to treat tumors in other portions of the body, such as prostate, breast, and brain tumors.

FIG. 8 illustrates an obstructing member similar to FIG. 7 with an orifice included to affect the release of the medicant. The orifice 114 of cavity cover 112 limits the release of medicant from cavity 110. Orifice 114 is sized and located to affect the release of medicant from the cavity 110.

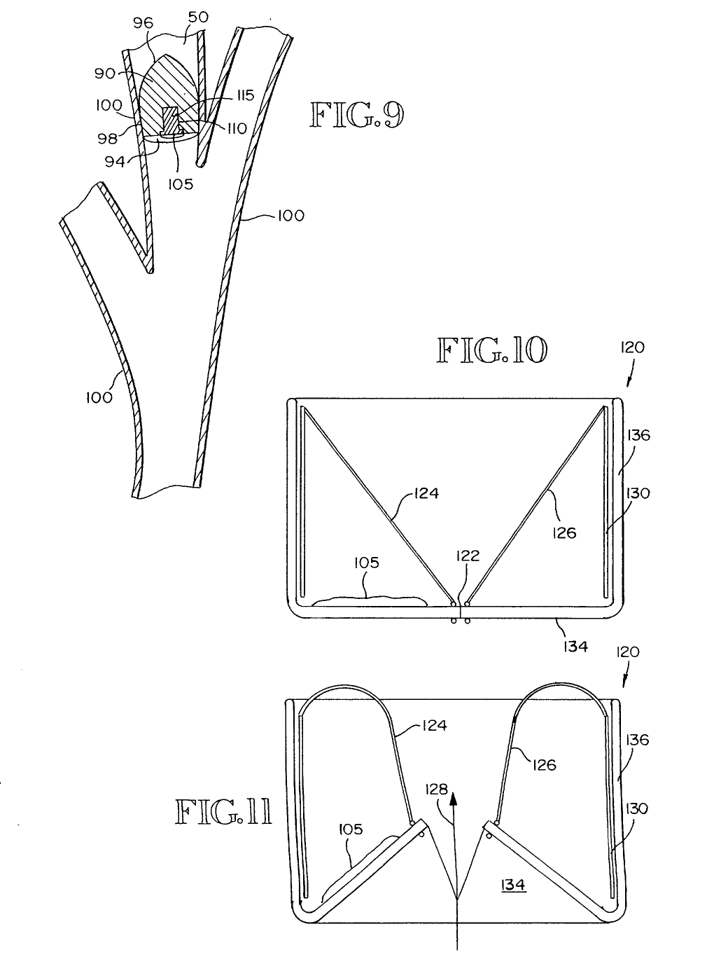

FIG. 9 is a longitudinal section view similar to FIG. 7 illustrating an alternative embodiment wherein the cavity 110 of obstructing member 90 includes an absorptive member 115 which carries a medicant 105. The absorptive member 115 may occupy all or at least a portion of the cavity 110. The absorptive member 115 may be any material and any configuration known to those skilled in the art, and as required by biological action desired and the limitations of selected medicant 105.

The embodiments of the invention illustrated in FIGS. 7-9 provide for associating medicant 105 with obstructive member 90 both before and/or after insertion into air passageway 50. This allows medicant 105 to be renewed after insertion, or to be initially associated after insertion. To that end, after insertion, a catheter could be used as generally illustrated in FIGS. 2 and 3 to access obstructive member 90. Medicant 105 could then be placed into cavity 110 of FIG. 7, or released for absorption into absorptive member 115 of FIG. 9.

FIGS. 10 and 11 illustrate a method in which localized control of biological interaction may be obtained according to a further embodiment of the invention. Here, the obstructing member 120 of the intra-bronchial device takes the form of a one-way valve. The one-way valve obstructing member 120 includes a generally circular base 134 and a circumferential generally cylindrical sidewall 136. Obstructing member 120 further includes resilient reinforcement rib 130. To form the valve, the base 134 includes a slit 122 to form a valve structure. On either side of the slit 122 is a tether 124 and 126, which extend to the resilient reinforcement rib 130. As illustrated in FIG. 11, obstructing member 120 is configured to be placed in an air passageway so that the one-way valve structure opens to permit airflow in the direction indicated by arrow 128, but precludes airflow in the opposite direction. When placed in an air passageway in an orientation that precludes inspiration and permits exhaustion to treat COPD, the valve action permits air to be exhaled (arrow 128) from the lung portion to be collapsed but precludes air from being inhaled into the lung portion to be collapsed.

In addition to generalized control of biological interaction, localized control of biological interaction with an intra-bronchial device may be provided by associating medicant 105 with a selected portion of an obstructive member, such as the one-way valve obstructing member 120. For example, fibrotic tissue might tend to grow across slit 122 and prevent the one-way valve structure from functioning. Medicant 105 may be selected to suppress such a fibrotic response, and associated with one-way valve obstructing member 120 in any manner previously described. As illustrated in FIGS. 10 and 11, for example, medicant 105 is associated with one-way valve obstructing member 120 by overlying a portion of a proximal surface of base 134 that forms the valve structure. The medicant 105 is thereby associated with a portion of base 134, and provides localized suppression of fibrotic response that otherwise might interfere with the functionality of the one-way valve structure.

Another aspect of the invention provides for targeted expression of biological response by a selected medicant. For example, a particular medicant may be selected to promote tissue granulation. Such tissue granulation may be desired to assist in device anchoring. The medicant 105 would be associated with the device at a site, such as the outer surface of the sidewall 136, where tissue granulation would assist in the anchoring of the obstructing member 120 to an air passageway.

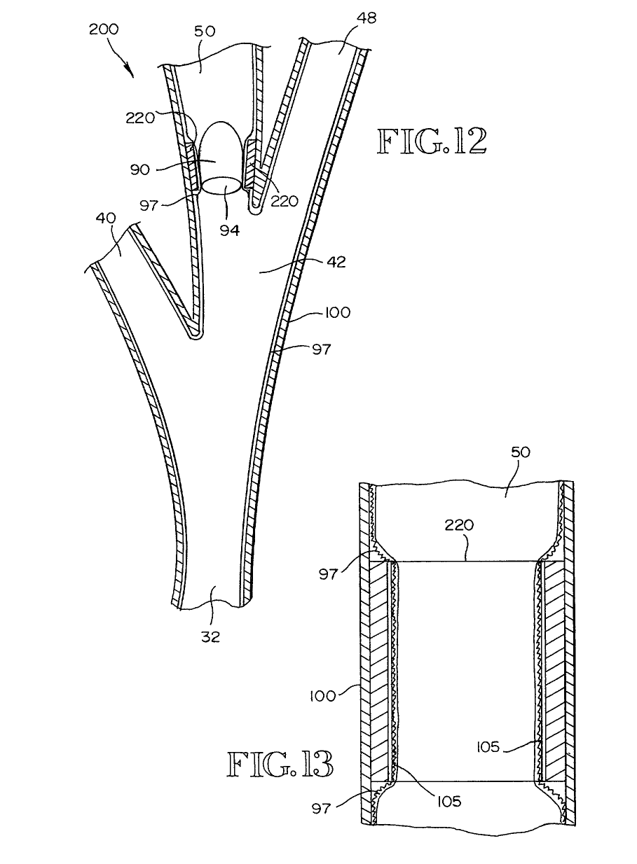

FIGS. 12 and 13 illustrate the use of a medicant to encourage a targeted expression of a biological response for an anchored intra-bronchial device in accordance with the present invention. FIG. 12 illustrates an intra-bronchial device 200 that includes an obstructing member 90 carried on a stent-like anchor 220 having a tubular shape. FIG. 12 further illustrates the stent-like anchor 220 and the obstructing member 90 positioned within air passageway 50. The stent-like anchor 220 and obstructing member 90 may each be made of any compatible materials and in any configuration known in the art suitable for placement in an air passageway by any suitable technique known in the art. Stent-like anchor 220 is anchored on bronchial wall 100 by a forced fit. To that end, the stent-like anchor 220 may be balloon expandable as is known in the art, or may be self-expanding. In a preferred embodiment, stent-like anchor 220 and obstructing member 90 are coupled before placement into air passageway 50. They may be coupled by any means appropriate for the materials used, method of installation selected, patient requirements, and degree of permanency selected. Coupling methods may include friction, adhesive and mechanical joint. In an alternative embodiment, stent-like anchor 220 and obstructive member 90 may be coupled during placement in air passageway 50.

FIG. 13 illustrates the stent-like anchor 220 disposed on bronchial wall 100, with obstructing member 90 omitted for clarity. Initially, the physical characteristics of stent-like anchor 220 may block the epithelial membrane 97. FIG. 13 illustrates the body's normal process of re-epithelialization. Epithelial membrane 97 and cilia will grow on stent-like anchor 220 over time, and permit mucus transport.

The effectiveness of intra-bronchial device 200 may depend in part on the anchor 220 being retained in the air passageway and the growth of the epithelial membrane 97 on the interior portion of the anchor 220. A medicant 105 selected to promote tissue granulation may be associated with the anchor 220 to assist in anchoring intra-bronchial device 200. Further, a medicant 105 selected to promote growth of epithelial membrane 97 on the interior may also be associated with the anchor 220 to assist with re-epithelialization.

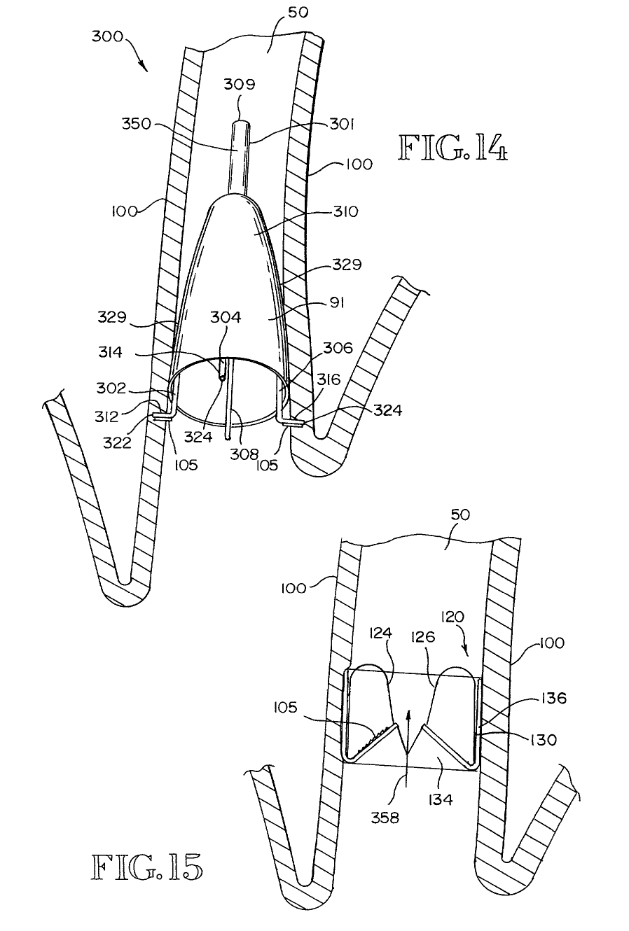

FIG. 14 illustrates the use of a medicant to encourage a targeted expression of a biological response for another embodiment of an anchored intra-bronchial device, in accordance with the present invention. Intra-bronchial device 300 is illustrated in a longitudinal cross-sectional view of air passageway 50 and anchored to air passageway wall 100. Intra-bronchial device 300 includes obstructing member 310 and anchoring device 350. Obstructing member 310 is anchored to the air passageway wall 100 by the anchoring device 350. Anchoring device 350 includes projections 312, 314, 316, and 318 that engage the air passageway wall 100 by piercing. Piercing anchors the obstructing member 90 to the air passageway wall 100, allowing it to resist movement such as might result from coughing or sneezing.

The piercing by projections 312, 314, 316, and 318 into the air passageway wall 100 may result in adverse effects on the patient or the performance of the intra-bronchial device 300, such as infection, inflammation, or rejection. A medicant 105 may be selected and associated with intra-bronchial device at projections 312, 314, 316, and 318, or elsewhere, to control any adverse biological interaction, or to encourage a biological reaction to retain projections 312, 314, 316, and 318 in place.

FIG. 15 illustrates a longitudinal cross-section view of the intra-bronchial device of FIGS. 10 and 11 placed in air passageway 50 to provide a medicant 105 to a patient, in accordance with the present invention. An embodiment of the invention provides for treating a disease or condition in the patient, particularly those associated with the lungs, by release of a medicant from an intra-bronchial device. An intra-bronchial device placed in air passageway 50 provides medicant 105 for intra-bronchial delivery to the patient.

The one-way valve obstructing member 120 of FIGS. 10 and 11 may be placed in air passageway 50 to provide medicant to a patient. Obstructing member 120 may be oriented in the air passageway 50 with the one-way valve orientated in either direction. FIG. 15 illustrates the one-way valve of obstructing member 120 orientated to permit inspiration airflow. The one-way valve structure opens to permit inspiration airflow in the direction indicated by arrow 358, but precludes exhaustion airflow. This orientation permits air to be inhaled into the distal lung portion, which may assist in delivering the medicant 105 to the distal lung portion communicating with the air passageway 50. Conversely, the one-way valve may be orientated to permit exhaustion airflow but preclude inspiration, if advantageous.

When treating chronic or acute pneumonia, the treatment objective may be to provide medicant 105 to the involved lung portion communicating with air passageway 50. An aspect of the invention provides for arranging and carrying medicant 105 on a distal portion of obstructing member 120 in a manner to promote intra-bronchial delivery. FIG. 15 illustrates medicant 105 carried on a distal portion of base 134 of obstructing member 120, which also forms a moveable part of the valve. In this structural arrangement, medicant 105 is physically exposed to the targeted distal lung portion, and movement of the valve with inhalation and against expiration may aid release of medicant 105. The structure of obstructing member 120 will substantially preclude the released medicant 105 from moving proximally, although some medicant 105 may move proximal to the obstructing member by escaping through the valve, between the wall 100 and obstructing member 120, or by mucociliary transport.

While the intra-bronchial device providing medicant 105 is illustrated in FIG. 15 as an obstructing member having a one-way valve, any form of intra-bronchial device may be used to provide medicant 105 to the patient. In an alternative embodiment, the intra-bronchial device carrying medicant 105 may be a member that does not obstruct, that only partially obstructs, or completely obstructs air passageway 50. For example, the intra-bronchial device carrying medicant 105 may be a tubular member, which may be balloon expandable as is known in the art, or may be self-expanding.

Intra-bronchial devices having other structures may be used to provide medicant 105 to the patient, and particularly to the lung portion communicating with the air passageway. For example, conical shaped obstructing member 90 of FIGS. 6-8 may be used. If the medicant is targeted for intra-bronchial delivery to the lung portion communicating with the air passageway 50, the orientation of obstructing member 90 may be with base 94 and medicant 105 toward the lung portion.

As can thus be seen from the foregoing, the present invention provides an intra-bronchial device and method for providing a medicant intra-bronchially. The medicant may be used for controlling biological interaction of an intra-bronchial obstruction device with the patient. The medicant may also be used to treat a disease or condition of the lungs. The medicant is provided by associating a medicant with the intra-bronchial obstruction device, either before, at the time of placement, or after placement.

While particular embodiments of the present invention have been shown and described, modifications may be made, and it is therefore intended in the appended claims to cover all such changes and modifications which fall within the true spirit and scope of the invention.

What is claimed is:

1. An intra-bronchial device adapted to be placed in an air passageway of a patient to collapse a lung portion communicating with the air passageway, the device comprising:
an obstructing member that prevents air from being inhaled into the lung portion to collapse the lung portion; and
a medicant carried by the obstructing member that controls biological interaction of the device with the patient.

2. The device of claim 1, wherein the medicant overlies at least a portion of the obstructing member.

3. The device of claim 1, wherein the medicant is imbedded in at least a portion of the obstructing member.

4. The device of claim 1, wherein the medicant is absorbed in at least a portion of the obstructing member.

5. The device of claim 1, wherein the obstructing member further includes an absorptive member and the medicant is absorbed by the absorptive member.

6. The device of claim 1, wherein the medicant is selected from a group consisting of tissue growth inhibitors, tissue growth enhancers, anti-microbial agents such as antibiotic agents or antibacterial agents, anti-inflammatory agents, and biological reaction inhibitors.

7. The device of claim 1, wherein the medicant is arranged to control biological interaction over a period of time.

8. The device of claim 1, wherein the medicant is co-mixed with at least a portion of the obstructing member.

9. An intra-bronchial device adapted to be placed in an air passageway of a patient to collapse a lung portion communicating with the air passageway, the device comprising:
an obstructing member that prevents air from being inhaled into the lung portion to collapse the lung portion;
a medicant that controls biological interaction of the device with the patient; and
a cavity in the obstructing member carrying the medicant.

10. The device of claim 9, wherein the medicant is selected from a group consisting of tissue growth inhibitors, tissue growth enhancers, anti-microbial agents such as antibiotic agents or antibacterial agents, anti-inflammatory agents, and biological reaction inhibitors.

11. The device of claim 9, wherein the medicant is arranged to control biological interaction over a period of time.

12. The device of claim 9, wherein the cavity further includes an absorptive member and the medicant is absorbed by the absorptive member.

13. The device of claim 9, wherein the cavity includes a cover having an orifice.

14. An intra-bronchial device for placement in an air passageway of a patient to collapse a lung portion communicating with the air passageway, the device comprising:
an obstructing member that prevents air from being inhaled into the lung portion to collapse the lung portion;
a medicant that controls biological interaction of the device with the patient; and
a support structure that is associated with the obstructing member and that carries the medicant.

15. The device of claim 14, wherein the medicant overlies at least a portion of the intra-bronchial device.

16. The device of claim 14, wherein the medicant is imbedded in at least a portion of the intra-bronchial device.

17. The device of claim 14, wherein the medicant is absorbed in at least a portion of the intra-bronchial device.

18. The device of claim 14, wherein the medicant is selected from a group consisting of tissue growth inhibitors, tissue growth enhancers, anti-microbial agents such as antibiotic agents or antibacterial agents, anti-inflammatory agents, and biological reaction inhibitors.

19. The device of claim 14, wherein the medicant is arranged to control biological interaction over a period of time.

20. A method of reducing the size of a lung of a patient using an intra-bronchial device while controlling biological interaction of the device with the patient, the method including the steps of:
providing an intra-bronchial device that precludes air from being inhaled through an air passageway into a lung portion to be reduced in size when inserted into the air passageway communicating with the portion of the lung;
associating a medicant that controls the biological interaction with the intra-bronchial device; and
inserting the intra-bronchial device in the air passageway.

21. The method of claim 20, wherein the step of associating the medicant with the intra-bronchial device is performed before the step of implanting the device.

22. The method of claim 20, wherein the step of associating the medicant with the intra-bronchial device includes overlying at least a portion of the intra-bronchial device with the medicant.

23. The method of claim 20, wherein the step of associating the medicant with the intra-bronchial device includes impregnating at least a portion of the intra-bronchial device with the medicant.

24. The method of claim 20, wherein the intra-bronchial device includes an absorptive member, and wherein the step of associating the medicant with the intra-bronchial device includes absorption of the medicant by the absorptive member.

25. The method of claim 20, wherein the medicant is selected from a group consisting of tissue growth inhibitors, tissue growth enhancers, anti-microbial agents such as antibiotic agents or antibacterial agents, anti-inflammatory agents, and biological reaction inhibitors.

26. The method of claim 20, wherein the medicant is arranged to control biological interaction over a period of time.

27. The method of claim 20, including the further steps of providing a cavity in the intra-bronchial device for receiving the medicant, and associating the medicant with the cavity.

28. The method of claim 27, wherein the step of associating the medicant with the intra-bronchial device is performed before the step of implanting the device.

29. The method of claim 27, wherein the cavity includes an absorptive member, and wherein the step of associating medicant with the intra-bronchial device includes absorption of the medicant by the absorptive member.

30. The method of claim 20, wherein the medicant is selected from a group consisting of tissue growth inhibitors, tissue growth enhancers, anti-microbial agents such as antibiotic agents or antibacterial agents, anti-inflammatory agents, and biological reaction inhibitors.

31. The method of claim 20, wherein the medicant is arranged to control biological action over a period of time.

32. An intra-bronchial device to provide a medicant intra-bronchially to a patient, the device comprising:
an intra-bronchial member adapted to be placed in an air passageway; and
a medicant carried on the intra-bronchial member.

33. The intra-bronchial device of claim 32, further including a cavity in the intra-bronchial member, and the medicant is carried in the cavity.

34. The intra-bronchial device of claim 32, wherein the medicant is arranged for delivery to a lung portion communicating with the air passageway.

35. The intra-bronchial device of claim 32, wherein the medicant is selected from a group consisting of antibacterial agents, antiviral agents, anthelmintic agents, anti-inflammatory agents, antitumor agents, radioprotective agents, antioxidant agents, adrenergic agents, hormonal agents, and radioactive branchytherapy material.

36. An intra-bronchial device adapted to be placed in an air passageway and provide a medicant to a patient, the device comprising:
an obstructing member that prevents air from being exhaled from the lung portion communicating with the air passageway; and
a medicant carried on the obstructing member.

37. The device of claim 36, wherein the medicant is arranged for delivery to the lung portion.

38. The device of claim 36, wherein the medicant is carried on a portion of the obstructing member exposed to the lung portion.

39. The device of claim 36, wherein the obstructing member when deployed in the air passageway substantially precludes released medicant from moving proximal to the obstructing member.

40. The device of claim 36, wherein the medicant overlies at least a portion of the obstructing member.

41. The device of claim 36, wherein the medicant is imbedded in at least a portion of the obstructing member.

42. The device of claim 36, wherein the medicant is absorbed in at least a portion of the obstructing member.

43. The device of claim 36, wherein the obstructing member further includes an absorptive member and the medicant is absorbed by the absorptive member.

44. The device of claim 36, wherein the medicant is selected from a group consisting of antibacterial agents, antiviral agents, anthelmintic agents, anti-inflammatory agents, antitumor agents, radioprotective agents, antioxidant agents, adrenergic agents, hormonal agents, and radioactive branchytherapy material.

45. The device of claim 36, wherein the medicant is arranged to control biological action over a period of time.

46. The device of claim 36, wherein the medicant is co-mixed with at least a portion of the obstructing member.

47. An intra-bronchial device adapted to be placed in an air passageway and provide a medicant to a patient, the device comprising:
an obstructing member that prevents air from being exhaled from the lung portion communicating with the air passageway;
a medicant; and
a cavity in the obstructing member carrying the medicant.

48. The intra-bronchial device of claim 47, wherein the cavity further includes an absorptive member and the medicant is absorbed by the absorptive member.

49. The intra-bronchial device of claim 47, wherein the cavity includes a cover having an orifice affecting release of the medicant.

50. The device of claim 47, wherein the medicant is exposed to the lung portion.

51. The device of claim 47, wherein the obstructing member when deployed in the air passageway substantially precludes released medicant from moving proximal to the obstructing member.

52. An intra-bronchial device adapted to be placed in an air passageway and provide a medicant to a patient, the device comprising:
an obstructing member that prevents air from being exhaled from the lung portion communicating with the air passageway;
a medicant; and
a support structure that is associated with the obstructing member and that carries the medicant.

53. A method of providing a medicant to a patient using an intra-bronchial device, the method including the steps of:
providing an intra-bronchial device for insertion into an air passageway in communication with a lung portion;
associating a medicant with the intra-bronchial device; and
inserting the intra-bronchial device in the air passageway.

54. The method of claim 53, wherein the intra-bronchial device precludes air from being exhaled through the air passageway when inserted into the air passageway.

55. The method of claim 53, wherein the medicant is an agent for treating a disease of the lungs, and the medicant is provided to treat a disease in the lung portion.

56. The method of claim 53, wherein the medicant is an agent for treating pneumonia, and the medicant is provided to treat pneumonia in the lung portion.

57. The method of claim 53, wherein the medicant is a radioactive material for treating cancer, and the medicant is provided to treat a cancer.

58. The method of claim 53, wherein the medicant is a radioactive material for treating cancer, and the medicant is provided to treat a cancer in the lung portion.

59. A device for reducing the size of a lung of a patient, the device comprising:
obstructing means for obstructing an air passageway communicating with a portion of the lung to be reduced in size, the obstructing means being dimensioned for insertion into the air passageway and for precluding air from being inhaled through the air passageway into the lung portion; and
means for controlling biological action of the obstructing means with the patient.

60. An intra-bronchial device adapted to be placed in an air passageway and provide a medicant to a patient, the device comprising:
obstructing means for obstructing an air passageway that prevents air from being exhaled from the lung portion communicating with the air passageway, the obstructing means being dimensioned for insertion into the air passageway and for precluding air from being exhaled through the air passageway into the lung portion; and
means for providing a biological action in the patient carried by the obstructing means.

61. The intra-bronchial device of claim 32, wherein the intra-bronchial member is arranged to preclude air movement in at least one direction.

62. The intra-bronchial device of claim 32, wherein the medicant overlies at least a portion of the intra-bronchial member.

63. The intra-bronchial device of claim 32, wherein the medicant is imbedded in at least a portion of the intra-bronchial member.

64. The intra-bronchial device of claim 32, wherein the medicant is absorbed in at least a portion of the intra-bronchial member.

65. The intra-bronchial device of claim 32, wherein the intra-bronchial member further includes an absorptive member and the medicant is absorbed by the absorptive member.

66. The intra-bronchial device of claim 32, the medicant is co-mixed with at least a portion of the intra-bronchial member.

67. The intra-bronchial device of claim 32, wherein the medicant is arranged to be released over a period of time.
